(19) United States
(12) Patent Application Publication
(10) Pub. No.: US 2005/0096689 A1
(43) Pub. Date: May 5, 2005

(54) Facial contouring device

(76) Inventor: Noga Yacoby, Tarzana, CA (US)

Correspondence Address: James E. Brunton, Esquire, Post Office Box 29000, Glendale, CA, 91209-9000, US

(21) Appl. No.: 10/702,873

(22) Filed: Nov. 5, 2003

(51) Int. Cl. A61M 29/00

(52) U.S. Cl. 606/191

(57) ABSTRACT

A facial contouring device for strategically contouring facial tissue located on either side of the user's mouth and nose to remove unsightly wrinkles. The device is preferably constructed from a fibrous material such as cotton that can be formed into a compressably deformable body which can be conveniently encapsulated within a sanitary, thin film covering. The film covering is constructed to carry an adhesive that functions to removably retain the device comfortably in position within the user's mouth.

### BACKGROUND OF THE INVENTION

1. Field of the Invention

The present invention relates generally to facial contouring devices. More particularly, the invention concerns a device that can be strategically positioned within the user's mouth to exert outward, wrinkle-smoothing forces on the facial tissue on either side of the mouth and nose.

2. Discussion of the Prior Art

Various methods and devices have been suggested in the past for wrinkle removal. The most common of these is plastic surgery. Such surgery is typically complex, expensive and, on occasion, fails to produce the desired results.

Also relatively common in the prior art are several types of wrinkle removing creams and like topical preparations. Such preparations are generally of limited value.

Additionally, a number of skin stretching devices have been suggested that purport to remove wrinkles by mechanically pulling loose facial skin taut. As a general rule, these devices are applied externally of the patient's face and employ various types of mechanical skin stretching mechanisms. Such devices are often unsightly and uncomfortable in use.

As will be better understood from the discussion which follows, the devices of the present invention are adapted to be conveniently inserted into the user's mouth and are specially configured to exert gentle, facial contouring forces on the skin in the area of the wrinkles which tend to controllably smooth the skin in a manner to eliminate or significantly reduce facial wrinkles. In the preferred form of the invention the devices include a compressably deformable body constructed from a multiplicity of co-operating filaments or fibrils such as cotton or synthetic filaments. The body of the device is incapsulated within a thin film covering, which carries an adhesive that removably positions the device between the user's gums and facial tissue to be contoured.

### SUMMARY OF THE INVENTION

It is an object of the present invention to provide a facial contouring device for strategically contouring facial tissue located on either side of the user's mouth and nose to remove unsightly wrinkles.

Another object of the invention is to provide a device of the aforementioned character which is easy to use and can be inexpensively manufactured.

Another object of the invention is to provide a facial contouring device that is adapted to be inserted into the user's mouth in a manner to controllably exert a gentle, externally directed skin smoothing pressure on the facial tissue in the area of the unsightly wrinkles.

Another object of the invention is to provide a facial contouring device of the character described in the preceding paragraphs that can be simply and economically produced so that it can be conveniently discarded after each treatment.

Another object of the invention is to provide a facial contouring device of the type described in the preceding paragraph that is constructed from a fibrous material such as cotton that can be formed into a compressably deformable body which can be conveniently encapsulated within a sanitary, thin film covering.

Another object of the invention is to provide a facial contouring device of the character described in which the film covering is constructed to carry an adhesive that functions to removably retain the device comfortably in position within the user's mouth.

The foregoing objectives, as well as various other objectives, are achieved by the devices of the invention, the details of construction of which are set forth in the paragraphs, which follow.

### BRIEF DESCRIPTION OF THE DRAWINGS

FIG. 1 is a generally illustrative view illustrating the wrinkle lines sought to be eliminated by the apparatus of the present invention.

FIG. 2 is a front view of one form of the facial forming device of the apparatus of the invention.

FIG. 3 is a rear view of the apparatus shown in FIG. 2.

FIG. 4 is a greatly enlarged, cross-sectional view taken along lines 4-4 of FIG. 2.

FIG. 5 is a greatly enlarged, cross-sectional view taken along lines 5-5 of FIG. 3.

FIG. 6 is a front view of an alternate form of the apparatus of the invention.

FIG. 7 is a rear view of the apparatus of the invention shown in FIG. 6.

FIG. 8 is a greatly enlarged, cross-sectional view taken along lines 8-8 of FIG. 7.

FIG. 9 is a generally perspective view of still an alternate form of the facial forming device of the apparatus of the invention.

### DESCRIPTION OF THE INVENTION

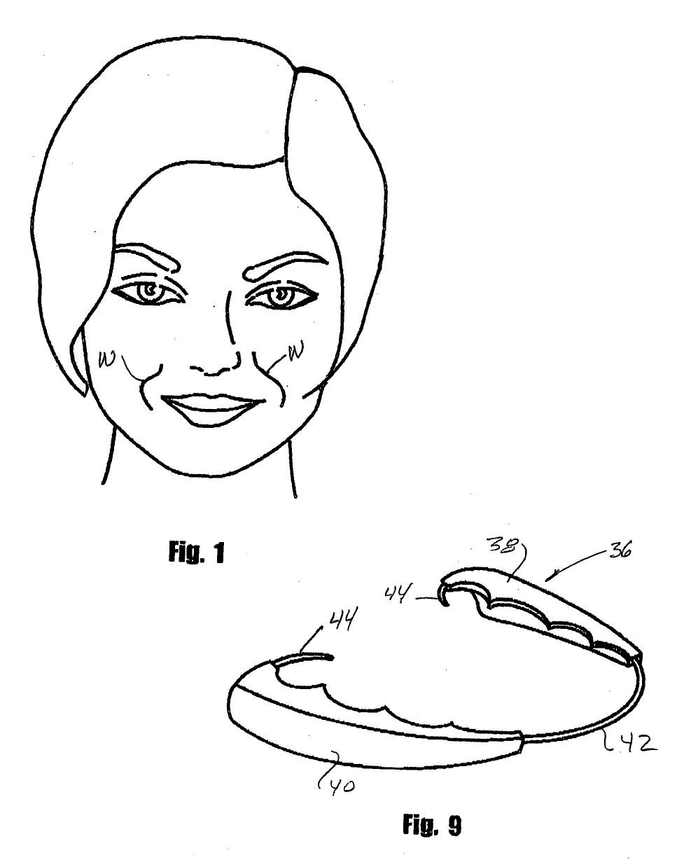

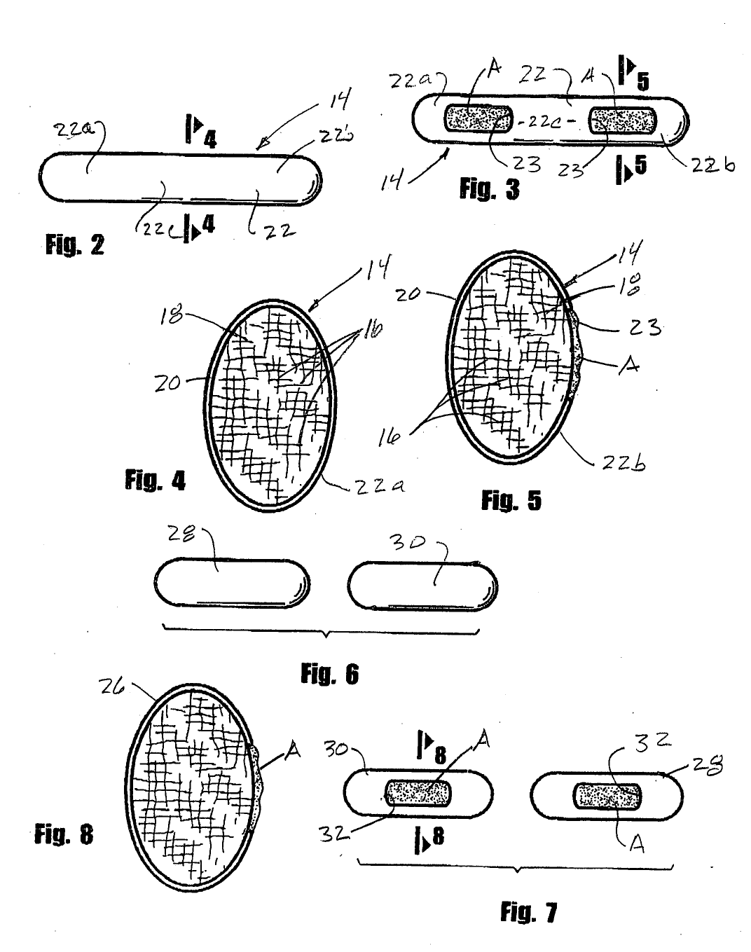

Referring to the drawings and particularly to FIGS. 1 through 5, one form of the facial forming device of the present invention is there illustrated. In this form of the invention the device comprises a uniquely configured, facial forming device 14 that is adapted to be removably positioned within the user's mouth. As best seen in FIGS. 2 through 5, the device 14 here comprises a yieldably deformable, fibrous body 18 that is made up of a multiplicity of fibrils or filaments 16 which co-operate to form the body 18. As best seen in FIGS. 4 and 5, yieldably deformable body 18 is substantially encapsulated by a thin film, plastic sheath 20 to form an elongated, wrinkle smoothing, facial contouring body 22 having first and second facial contouring portions 22a and 22b and a central portion 22c which spans the user's mouth. As illustrated in FIGS. 3 and 5, plastic sheath 20 is provided with a pair of spaced-apart openings 23 the purpose of which will presently be described.

To hold the elongated facial contouring body 22 in position within the user's mouth, bonding means, here provided in the form of a suitable adhesive “A” of a character well known to those skilled in the art, is disposed within the openings 23. During use, the elongated facial contouring body 22 is inserted into the user's mouth between the gums and the upper lip so that the facial contouring portions 22a and 22b of the device are located immediately below the wrinkles “W” (FIG. 1) that are formed on either side of the user's nose and mouth. When the device is in this position, the central portion 22c of the device resides below the user's nose and the yieldably deformable fibrous body portions of the device exert a gentle, wrinkle smoothing outwardly directed force on the facial tissue which tends to effectively smooth out the wrinkles “W”. Following this wrinkle smoothing operation, the device of the invention can be removed from the user's mouth and conveniently discarded.

While the fibrous body portion 18 of the device can be constructed from various types of fibrous material, a readily commercially available cotton fibrous material has proven satisfactory for use in constructing the device of the invention. More particularly, experience has shown that the cotton fibrils 16 can readily be formed into the generally oval shaped configuration shown in FIGS. 4 and 5 and can be retained in the desired configuration by the plastic sheath 20. However, it is to be understood that fibrous materials such as other natural and synthetic materials can be used to satisfactorily form body 18.

Turning next to FIGS. 6 through 8, an alternate form of the facial forming device of the invention is therein shown. This form of the invention is similar in many respects to that described in connection with FIGS. 2 through 5. However, in this latest form of the invention, the fibrous body portion of the device is constructed in two separate parts rather than as a single elongated body portion. As indicated in FIG. 8 each of the separate body portion parts is substantially encapsulated by a thin film sheet generally designated by the numeral 26. As depicted in FIGS. 6 and 8, each of the facial contour forming portions 28 and 30 of this latest form of the invention is generally oval shaped in cross-section.

As in the earlier described form of the invention, each of the thin film sheets 26 that encapsulate the two body portions 28 and 30 is provided with an opening 32 that carries the adhesive “A” that functions to retain the body portions in position on either side of the user's mouth and directly below the wrinkles “W” (FIG. 1). With the body portions 28 and 30 in this position, a gentle outward force will be directed against the facial tissue on either side of the user's mouth tending to efficiently smooth the facial tissue in the area of the wrinkles “W”.

Turning lastly to FIG. 9, still another embodiment of the invention is there illustrated and generally designated by the numeral 36. Unlike the earlier described forms of the invention, this latest embodiment comprises a custom made facial forming device that can be removably positioned within the user's mouth. Device 36 comprises an assembly made up of first and second molded plastic, facial contouring portions 38 and 40 that are custom formed for the particular user. Portions 38 and 40 are suitably interconnected by connector means shown here as a flexible wire connector 42.

Also comprising a part of this latest embodiment of the invention is retaining means for removably holding the wrinkle smoothing body portions 38 and 40 in position within the user's mouth. This retaining means here comprises a pair of strategically curved, wire gripping segments 44 that are connected to the first and second facial contouring portions 38 and 40 in the manner illustrated in FIG. 9. Gripping segments 44 are custom configured to securely gripped selected teeth within the user's mouth.

1. A facial forming device removably positioned within a user's mouth, said device comprising:
(a) a multiplicity of filaments co-operating to form a yieldably, deformable body;
(b) a thin film sheath substantially encapsulating said body to form a wrinkle smoothing body, said sheath having at least one opening formed therein; and
(c) bonding means disposed within said opening in said thin film sheath for removably holding said wrinkle smoothing body in position within the user's mouth.

2. The device as defined in claim 1 in which said filaments comprise cotton filaments.

3. The device as defined in claim 1 in which said bonding means comprises an adhesive.

4. The device as defined in claim 1 in which the user's mouth includes first and second sides and in which said wrinkle smoothing body includes a first facial contouring portion positioned within the first side of the user's mouth and a second facial contouring portion positioned within the second side of the user's mouth.

5. The device as defined in claim 4 in which said wrinkle smoothing body further includes a central portion interconnecting said first and second facial contouring portions.

6. The device as defined in claim 4 in which said first and second facial contouring portions are generally oval in cross-section.

7. A facial forming device removably positioned within a user's mouth, said device comprising:
(a) a multiplicity of filaments co-operating to form a yieldably, deformable body having first and second spaced apart portions;
(b) a thin film sheath substantially encapsulating said yieldably deformable body to form a facial contouring body, said sheath having a plurality of openings formed therein; and
(c) an adhesive carried within said openings formed and said thin film sheath for removably holding said facial contouring body in position within the user's mouth.

8. The device as defined in claim 7 in which said yieldably deformable body further includes a central portion interconnecting said first and second spaced apart portions.

9. The device as defined in claim 8 in which said first and second facial contouring portions are generally oval in cross-section.

10. A facial forming device removably positioned within a user's mouth, said device comprising:
(a) a cotton fibrous material molded to form a yieldably, deformable, wrinkle smoothing body having first and second facial contouring portions;
(b) a thin film sheath substantially encapsulating said yieldably deformable body to form a facial contouring body, said sheath having first and second in openings formed therein; and
(c) bonding means comprising an adhesive material disposed within said openings for removably holding said facial contouring body in position within the user's mouth.

11. A facial forming device removably positioned within a user's mouth, said device comprising:
(a) first and second spaced apart molded plastic facial contouring portions;
(b) connector means for interconnecting said first and second molded plastic facial contouring portions; and
(c) retaining means for removably holding said first and second spaced apart molded plastic facial contouring portions in positioned within the user's mouth.

12. The device as defined in claim 11 in which said retaining means comprises wire gripping segments connected to said first and second molded plastic facial contouring portions for gripping selected teeth within the user's mouth.
